(19) United States
(12) Patent Application Publication
(10) Pub. No.: US 2026/0240973 A1
(43) Pub. Date: Aug. 20, 2026

(54) Use of O-Antigens for Treating Bacterial Infections

(71) Applicant: Agency for Science, Technology and Research, Singapore (SG)

(72) Inventors: Lai Guan Ng, Singapore (SG); Shu Zhen Chong, Singapore (SG); Ye Chean Teh, Singapore (SG); Ming Yao Chooi, Singapore (SG); Swaine Chen, Singapore (SG)

(73) Assignee: Agency for Science, Technology and Research, Singapore (SG)

(21) Appl. No.: 19/161,632

(22) PCT Filed: Mar. 3, 2023
(86) PCT No.: PCT/SG2023/050127

§ 371 Date: Sep. 2, 2025

(51) Int. Cl. A61K 39/108; A61K 39/00; A61P 37/04

(52) U.S. Cl. CPC A61K 39/0258; A61P 37/04; A61K 2039/572

(57) ABSTRACT

The present invention relates to uses of O-antigens for treating bacterial infections. In one aspect of the present invention, there is provided a use of an O-antigen in the manufacture of a medicament for treating a bacterial infection, wherein the medicament to be administered mobilises transitional premonocytes (TpMos) from a bone marrow into a peripheral blood of a subject. In another aspect, there is provided an O-antigen for use in treating a bacterial infection, wherein the O-antigen mobilises TpMos from a bone marrow into a peripheral blood of a subject. In another aspect, there is provided a method of mobilizing proliferating transitional premonocytes (TpMos) from a bone marrow into a peripheral blood of a subject, the method comprising administering to the subject a therapeutically effective amount of an O-antigen.

### FIELD OF THE INVENTION

The present invention generally relates to the treatment of bacterial infections, and more particularly relates to the use of O-antigens for treating bacterial infections.

### BACKGROUND

Sepsis is one of the leading causes of mortality in intensive care units. Intense research effort has focused on understanding the pathogenesis of sepsis and formulating intervention strategies such as antibiotic therapies and resuscitation using intravenous fluids. However, due to the complexity and rapidity of sepsis progression, current therapies and biomarkers for prediction of outcome remain limited in their success.

Despite the advances in supportive measures for sepsis, the mortality rate of sepsis remains high. As sepsis is often associated with bacterial infections, antibiotic treatments are usually administered to prevent detrimental outcomes. However, it is increasingly clear that broad spectrum antibiotic treatments are becoming less effective against bacteria and can cause adverse effects to the patients due to antimicrobial resistance which has emerged as a global threat to public health systems worldwide.

There is thus a need for new intervention strategies for the treatment of bacterial infections and sepsis that overcome the drawbacks of the prior art. Furthermore, other desirable features and characteristics will become apparent from the subsequent detailed description and the appended claims, taken in conjunction with the accompanying drawings and this background of the disclosure.

### SUMMARY

In one aspect, the present invention provides a use of an O-antigen in the manufacture of a medicament for treating a bacterial infection, wherein the medicament to be administered mobilises transitional premonocytes (TpMos) from a bone marrow into a peripheral blood of a subject.

In one embodiment, the bacterial infection is bacterial sepsis.

In one embodiment, the O-antigen is an O-antigen from E. coli.

In one embodiment, the O-antigen comprises the structure DGIc-(a1-4)-DGal-(a1-3)-DGIcNAc.

In one embodiment, the O-antigen is an O-antigen from E. coli O18 serotype or O111 serotype.

In one embodiment, the E. coli O18 serotype is E. coli UTI89 strain.

In one embodiment, the medicament is used as an adjunct treatment.

In another aspect, there is provided a pharmaceutical composition comprising (a) a therapeutically effective amount of an O-antigen comprising the structure DGIc-(α1-4)-DGal-(a1-3)-DGIcNAc; and (b) one or more pharmaceutically acceptable carriers and/or diluents.

In one embodiment, the O-antigen is an O-antigen from E. coli.

In one embodiment, the O-antigen is an O-antigen from E. coli O18 serotype or O111 serotype.

In one embodiment, the E. coli O18 serotype is E. coli UTI89 strain.

In another aspect, there is provided an O-antigen for use in treating a bacterial infection, wherein the O-antigen mobilises TpMos from a bone marrow into a peripheral blood of a subject.

In one embodiment, the bacterial infection is bacterial sepsis.

In one embodiment, the O-antigen is an O-antigen from E. coli.

In one embodiment, the O-antigen comprises the structure DGIc-(a1-4)-DGal-(a1-3)-DGIcNAc.

In one embodiment, the O-antigen is an O-antigen from E. coli O18 serotype or O111 serotype.

In one embodiment, the E. coli O18 serotype is E. coli UTI89 strain.

In one embodiment, the O-antigen is used as an adjunct treatment.

In one aspect, there is provided a method for treating a bacterial infection comprising administering to a subject a therapeutically effective amount of a composition comprising an O-antigen, wherein the administration of the O-antigen mobilises transitional premonocytes (TpMos) from a bone marrow into a peripheral blood of the subject.

In one embodiment, the bacterial infection is bacterial sepsis.

In one embodiment, the O-antigen is an antigen from E. coli.

In one embodiment, the O-antigen comprises the structure DGIc-(a1-4)-DGal-(a1-3)-DGIcNAc.

In one embodiment, the O-antigen is an O-antigen from E. coli O18 serotype or O111 serotype.

In one embodiment, the E. coli O18 serotype is E. coli UTI89 strain.

In one embodiment, the amount of O-antigen present in the composition is between 40 ng and 400 ng.

In one embodiment, the O-antigen is used as an adjunct treatment.

In one aspect, there is provided a method of mobilizing transitional premonocytes (TpMos) from a bone marrow into a peripheral blood of a subject, the method comprising administering to the subject a therapeutically effective amount of an O-antigen.

In one embodiment, the therapeutically effective amount of the O-antigen is between 40 ng and 400 ng of the O-antigen.

In one embodiment, the O-antigen is an O-antigen from E. coli.

In one embodiment, the O-antigen comprises the structure DGIc-(a1-4)-DGal-(a1-3)-DGIcNAc.

In one embodiment, the O-antigen is an O-antigen from E. coli O18 SEROTYPE OR O111 serotype.

In one embodiment, the E. coli O18 serotype is E. coli UTI89 strain.

In another aspect, there is provided a method of determining a prognosis of a subject having sepsis induced by a Gram-negative bacterial infection, the method comprising detecting a presence of transitional premonocytes (TpMos) in a blood sample obtained from the subject, wherein the presence of TpMos in the blood sample is indicative of a favourable clinical outcome.

Advantageously, the inventors of this invention have demonstrated that O-antigen mobilizes TpMos from a bone marrow into a peripheral blood of the subject which has therapeutic effects against sepsis.

### BRIEF DESCRIPTION OF THE DRAWINGS

The invention will be better understood with reference to the detailed description when considered in conjunction with the non-limiting examples and the accompanying drawings, in which:

FIG. 1 shows the emergence of Ly6C^{hi} proliferative monocytes identified as transitional pre-monocytes (TpMos) that have been mobilized from the bone marrow (BM) into the peripheral blood during bacterial infection and sepsis. (A-B) Representative fluorescence-activated cell sorting (FACS) plots (A) and number of blood proliferative Ly6C^{hi} monocytes (B) in PBS control and E. coli -infected mice that were incorporated BrdU in vivo. Results are expressed as mean (n=4) and representative of one of three experiments. *** P<0.001 (Student's t test). (C) Mice were subjected to CLP-induced sepsis, and proliferative Ly6C^{hi} monocytes in the blood were quantified on the basis of BrdU incorporation at indicated time points. Results are expressed as mean (n=5) and representative of one of three experiments. ** P<0.01, and **** P<0.0001 (one-way ANOVA). (D) Uniform Manifold Approximation and Projection (UMAP) analysis of monocyte subsets from uninfected BM cells (left), uninfected blood cells (middle), and E. coli -infected blood cells (right). Parameters used for UMAP projection include Ly6C, CXCR4, CD49f, CD115, cKit, CD43, CX3CR1, and CD48. Monocyte subsets, including proliferative (Fucci⁺) Ly6C^{hi} monocytes, were then manually gated and overlaid onto the UMAP space. (E) The number of BM TpMos (left) and blood Ly6C^{hi} proliferative monocytes (right) at indicated time points. Results are expressed as mean (n=4) and representative of one of two experiments. P<0.05, *** P<0.001, and **** P<0.0001 (one-way ANOVA). (F) GFP-tagged TpMos and tdTomato tagged-MatMos (mature monocytes) were resuspended in 1:1 ratio and transferred into recipient mice via the intra-femoral route (IBM). Recipient mice were then injected with PBS or E. coli intraperitoneally and harvested for analysis 9 hours after infection (left). Representative FACS plot of transferred cells showing incorporation of BrdU in vivo (middle). Percentage of BrdU positive cells among the transferred cells (right). Results are expressed as mean (n=4-5) and representative of one of three experiments. ** P<0.001 (one-way ANOVA).

FIG. 2 shows TpMos conferred protective responses and improved the survival rate of septic mice. (A) LPS-stimulated control BM TpMos and MatMos were analyzed for apoptotic cells using FLICA-polycaspase after 3 hours. Results are expressed as mean (n=4-6) and representative of one of three experiments. *** P<0.001 (Student's t-test). (B) TpMos and MatMos were sorted from CD45.1 mice and adoptively transferred into CLP-induced CD45.2 recipient mice via the intraperitoneal (i.p.) route. Transferred cells were analyzed on Day 1, 3, and 5 after the adoptive transfer for the expression of IL-6, IL-13, TNF-a, and iNOS. Results are expressed as mean (n=4-6) and representative of one of three experiments. n.s. not significant, *P<0.05, **P<0.01, ***P<0.001, ****P<0.0001 (Student's t-test). (C) TpMos or MatMos were sorted and adoptively transferred into recipient mice shortly after they were subjected to CLP. The survival rate of these mice was assessed using the Kaplan-Meier survival curve. Results are representative of one of three experiments (n=10. *P<0.05 (Mantel-Cox).

FIG. 3 shows mobilization of TpMo is specific to bacterial-induced inflammatory condition. Representative FACS plots showing TpMos (CD62L^{hi} CXCR4^{hi}) in CLP-induced sepsis model (A), malaria (B), influenza (C), dengue (DENV) and zika (ZIKV) model (D), high fat diet model (E), and pregnancy model (F).

FIG. 4 shows TpMo mobilization is induced by specific gram-negative bacteria O-antigen glycan structure. (A) The number of circulating TpMos in mice challenged with gram-negative bacteria (E. coli, A. baumannii, P. aeruginosa), and Gram-positive bacteria (S. aureus, E. faecalis, and E. faecium).Results are expressed as mean (n=4-5 per group). ****P<0.0001 (One-way ANOVA). (B) The number of circulating TpMos in mice challenged with E. coli strains of different O-antigen structure and densities (MG1655, L5, L9, UTI89). Results are expressed as mean (n=4-7 per group) and representative of one of two experiments. ****P<0.0001 (One-way ANOVA). (C) The number of circulating TpMos in mice challenged with LPS of different O-antigens (O55, O111). Results are expressed as mean (n=4 per group) and representative of one of two experiments. ****P<0.0001 (One-way ANOVA). (D) Common glycan structure between 018 and O 111 antigens which were able to induce TpMo mobilization is highlighted. O18A is a subtype of O18 serotype.

FIG. 5 shows briefly the development of monocytes in the bone marrow (BM). Monocytes are derived from the common monocyte progenitor (cMoP) through a transitional precursor before maturing into mature Ly6C^{hi} monocytes (MatMos). This transitional precursor was termed the transitional premonocytes (TpMos). TpMos are functionally immature, actively proliferating in the BM, do not respond to mobilization cues, and were thought to be a reserved reservoir of MatMos.

FIG. 6 shows the importance of infiltrating monocytes during bacterial infections. During inflammation, monocytes must be recruited to the inflamed site rapidly and differentiate into monocyte-derived macrophages to replenish the niche and provide effector functions such as phagocytosis and production of proinflammatory cytokines to reduce the bacterial load.

FIG. 7 shows phagocytosis of bacteria leads to monocyte exhaustion and cell death. Monocytes have lower mitochondria and ATP levels. Thus, the ingestion of bacteria often leads to monocyte exhaustion followed by cell death, limiting their capacity for subsequent macrophage differentiation.

FIG. 8 shows some key findings of TpMos. During bacterial infection and sepsis, TpMos could migrate as effectively as MatMos into the peritoneum and retained their proliferative capacity. TpMos displayed enhanced responses towards CSF-1 and generated more macrophages compared to MatMos. TpMos and TpMo-derived macrophages are more viable and less inflammatory compared to MatMos and MatMo-derived macrophages. In addition, TpMos did not contribute to cytokine storm.

FIG. 9 shows that TpMos can influence the outcome of sepsis. To determine if TpMos may be protective or detrimental during sepsis, TpMos and MatMos were sorted and adoptivedly transferred into septic mice and their survial was compared. In an aspect of the invention, the adoptive transfer of TpMos in a subject showed improved survival outcome of sepsis, suggesting that the mobilization of TpMos are protective for bacterial immune responses.

FIG. 10 shows the lipopolysaccharide (LPS) components. LPS represents a major part of the cell wall components of Gram-negative bacteria. It comprises of lipid A, core oligosaccharide, and O-polysaccharide (O-antigen). Different forms of LPS play a major role in bacterial virulence, permeability, and cell adhesion. Among the components that form the LPS structure, the O-antigens have been described to be involved in bacterial virulence.

FIG. 11 shows that the presence of O-antigen and serotype drives bacteria pathogenicity. Since O-antigens were described to be involved in bacterial virulence, E. coli strains with different O-antigen structures and pathogenicities were used to determine if O-antigen plays a role in TpMo mobilization. Data was obtained and studied for determining the effect of using O-antigen to mobilize TpMos as is described in this invention.

FIG. 12 shows that bacterial virulence plays a role in triggering TpMo release. In contrast to MatMos that did not show any trend in numbers associated with different strains at varying doses, TpMos were shown to be released into the circulation when the mice were infected with UTI89 carrying O18 antigen.

FIG. 13 TpMos are transitional precursors of mature Ly6C^{hi} monocytes (MatMos). During steady state, they are functionally immature, actively proliferating in the BM, and serve as a reservoir for MatMos in the BM.

FIG. 14 shows TpMos' behaviour and functions during severe imflammation. TpMos can leave the BM into the circulation and enter the inflamed site during bacterial infection and sepsis. TpMos replenish the macrophage pool actively and conferred protection in sepsis by balancing the pro-inflammatory cytokines produced by MatMos.

FIG. 15 shows sepsis intervention strategies.

FIG. 16 shows a general research and development plan in accordance with an embodiment of this invention.

FIG. 17 shows that TpMos are present in septic patient. Representative FACS plots showing the presence of TpMos (CXCR4^{pos} Ki67^{pos}) in the circulation of septic patient from ICU.

### DETAILED DESCRIPTION OF THE PRESENT INVENTION

In one aspect, the present invention provides a use of an O-antigen in the manufacture of a medicament for treating a bacterial infection, wherein the medicament to be administered mobilises transitional premonocytes (TpMos) from a bone marrow into a peripheral blood of a subject.

The term “O-antigen”, also known as “O-specific polysaccharide” or “O-side chain”, is meant to refer to a major component of the surface lipopolysaccharide (LPS) of Gram-negative bacteria that is highly variable in structure. As used herein, the “O-antigen” may include a reference to the O-antigen itself, the O-antigen as part of an intact lipopolysaccharide (LPS), or the O-antigen as part of an intact LPS on a bacterial cell.

As used herein, the term “treat” or “treating” in the context of treating a disease such as sepsis is meant to include improving clinical outcomes of patients having the disease. This includes improving the survival rates of patients having the disease.

As used herein, a transitional premonocyte (TpMo) is a cell that has the following surface markers: Ly6C^{hi}, CXCR4^{hi}, CCR2^{lo}, CD62L^{hi}, CD11b^{lo}, and CD31^{hi}. Transitional premonocytes (TpMos) are actively proliferating by nature. The term “proliferating” in the context of a cell refers to the cell being in the S/G2/M phase of the cell cycle. The terms “transitional premonocyte” and “proliferating transitional premonocyte” are used interchangeably. TpMos are in an active proliferation stage which can be shown by the expression of BrdU via BrdU assay, or by the expression of Fucci signal in Fucci-474 transgenic mouse which labels cells in the S/G2/M phase of the cell cycle with a green-emmiting fluorescent protein.

The term “bacterial infection” is meant to include a condition in which a subject is infected with bacteria. As used herein, the term “bacterial infection” is also used to describe disease states characterized by the presence of bacteria, such as sepsis. As used herein, the term “sepsis” refers to a life-threatening organ disfunction caused by a dysregulated host response to infection. Sepsis is most commonly caused by bacterial infections, but can also be caused by viral infections or fungal infections. The term “bacterial sepsis” as used herein refers to sepsis caused by bacteria.

The term “sepsis” is also used to refer to a deleterious systemic inflammatory response to infection, formally defined as the presence (probable or documented) of infection together with systemic manifestations of infection. The term “sepsis” as used herein encompasses complications thereof such as “severe sepsis” and “septic shock”.

The term “periphery” is meant to include the peripheral blood and tissue site of infection. Peripheral blood refers to blood that circulates through the body's blood vessel system and is not concentrated within a specific organ. The terms “mobilise into periphery” and “mobilise into circulation” are used interchangeably.

In one embodiment, the bacterial infection is bacterial sepsis.

In one embodiment, the O-antigen is an O-antigen from E. coli.

In one embodiment, the O-antigen comprises the structure DGIc-(a1-4)-DGal-(a1-3)-DGIcNAc.

In One Embodiment, the O-antigen Is an O-antigen From E. Coli O18 serotype or O111 serotype.

In one embodiment, the E. coli O18 serotype is E. coli UTI89 strain.

In one embodiment, the medicament is used as an adjunct treatment.

As used herein, the term “adjunct treatment” refers to a treatment that is used with a primary treatment to assist or enhance the primary treatment. The adjunct therapy may increase the effectiveness of the primary treatment in treating a condition. The terms “adjunct treatment”, “adjuvant treatment”, “adjunct therapy” and “adjuvant therapy” are used interchangeably. The term “treatment” may refer to the prophylactic and/or therapeutic treatment.

In another aspect, there is provided a pharmaceutical composition comprising (a) a therapeutically effective amount of an O-antigen comprising the structure DGIc-(a1-4)-DGal-(a1-3)-DGIcNAc; and (b) one or more pharmaceutically acceptable carriers and/or diluents.

As used herein, the term “pharmaceutical composition” is meant to include any pharmaceutical preparation or formulation which is suitable for administration to a subject in need thereof. The composition may be suitable for parenteral administration either naked or complexed with a delivery agent to a patient. The carrier is selected from the group consisting of a nanoparticle, such as a polymeric nanoparticle; a liposome, such as pH-sensitive liposome, an antibody conjugated liposome; a viral vector, a cationic lipid, a polymer, and a cell penetrating peptide. It will be appreciated that pharmaceutical compositions provided according to the disclosure may be administered by any means known in the art. The pharmaceutical composition may be administered orally, or rectal, or transmucosal, or intestinal, or intramuscular, or subcutaneous, or intramedullary, or intrathecal, or direct intraventricular, or intravenous, or intravitreal, or intraperitoneal, or intranasal, or intraocular.

A pharmaceutically acceptable carrier refers, generally, to materials that are suitable for administration to a subject wherein the carrier is not biologically harmful, or otherwise, causes undesirable effects. Such carriers are typically inert ingredients of a medicament. Typically a carrier is administered to a subject along with an active ingredient without causing any undesirable biological effects or interacting in a deleterious manner with any of the other components of a pharmaceutical composition in which it is contained. Suitable pharmaceutical carriers are described in Martin, Remington's Pharmaceutical Sciences, 18th Ed., Mack Publishing Co., Easton, Pa., (1990), incorporated by reference herein in its entirety.

The pharmaceutical composition may include a pharmaceutically acceptable salt, which may refer to the relatively non-toxic, inorganic and organic acid addition salts of compounds as described herein. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobsonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like (see, for example, Berge S. M, et al, “Pharmaceutical Salts,” J. Pharm. Sci., 1977;66:1-19 which is incorporated herein by reference).

The pharmaceutical formulations of the disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly bringing into association the active ingredients with liquid carriers or finely divided solid carriers or both, and then, if necessary, shaping the product.

Combination therapy with an additional therapeutic agent may also contemplated by the disclosure. The term “combination” or “combination therapy” as used throughout the specification, is meant to encompass the administration of the referred therapeutic agents to a subject suffering from a disease, disorder or pathological condition, in the same or separate pharmaceutical formulations, and at the same time or at different times. If the therapeutic agents are administered at different times they should be administered sufficiently close in time to provide for the potentiating or synergistic response to occur. In such instances, it is contemplated that one would typically administer both therapeutic agents within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In other situations, it might be desirable to reduce the time between administration, administering both therapeutic agents within seconds or minutes to hours, preferably within about 6 hours from each other, more preferably within about 1 or 3 hours. A combination therapy for use in the method of treatment of the invention may include the use of various classes of antibiotics, typically at least two different classes of antibiotics. This includes combinations of two or three antibiotics given concomitantly. Antibiotics typically used in the treatment of sepsis include aminoglycosides, macrolides, beta-lactams (e.g., penicillins; first, second, third and fourth generation cephalosporins, and carbapenems), glycopeptides, lincosamides and/or fluoroquinolones. For instance, a beta-lactam agent (e.g., a third or fourth generation cephalosporin) with a macrolide, fluoroquine or aminoglycoside is generally used for broad-spectrum coverage.

The term “therapeutically effective amount” refers to the amount of the O-antigen that is required to confer the intended therapeutic effect in the subject, which amount will vary depending on the route of administration, status of infection or sepsis, and possible inclusion of other therapeutics or excipients. The method and uses of the invention are for a patient in need thereof. The compositions and methods of this invention are for a subject or patient in need thereof. The term “patient in need thereof” refers to a person who has or is suspected of having or developing sepsis. The term “prevention of sepsis” means preventing or inhibiting the development of sepsis in a patient with a blood infection, or preventing or inhibiting the development of severe sepsis in a patient with a sepsis, or preventing or inhibiting the development of septic shock in a patient with severe sepsis.

To practice the methods of this invention, the O-antigen may be administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parentally acceptable diluents or solvent for example, as a solution in 1,3-butanediol. Among the acceptable vehicles and solvents that can be employed are mannitol, water, Ringer's Solution and isotonic sodium chloride solution. In addition, sterile, fixed oils are conventionally employed as a solvent or suspending medium (eg. Synthetic mono-or dyglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluents or dispersant, or carboxymethyl cellulose or similar dispersing agents. Other commonly used surfactants such as Tweens or Spans or other similar emulsifying agents or bioavailablity enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavouring, or colouring agents can be added. A nasal aerosol or inhalation composition can be prepared according to techniques well known in the art of pharmaceutical formulation. A fused multicyclic compound-containing composition can also be administered in the form of suppositories for rectal administration. The carrier in the pharmaceutical composition must be “acceptable” in the sense of being compatible with the active ingredient of the formulation (and preferable, capable of stabilising it) and not deleterious to the subject to be treated. For example, one or more solubilising agents, which form more soluble complexes with the fused multicyclic compounds, or more solubilising agents, can be utilised as pharmaceutical carriers for delivery of the active compounds. Examples of other carriers include colloidal silicon dioxide, magnesium stearate, sodium lauryl sulphate, and D&C Yellow #10.

In one embodiment, the O-antigen is an O-antigen from E. coli.

In one embodiment, the O-antigen is an O-antigen from E. coli O18 serotype or O111 serotype.

In one embodiment, the E. coli O18 serotype is E. coli UTI89 strain.

In another aspect, there is provided an O-antigen for use in treating a bacterial infection, wherein the O-antigen mobilises TpMos from a bone marrow into a peripheral blood of a subject.

In one embodiment, the bacterial infection is bacterial sepsis.

In one embodiment, the O-antigen is an O-antigen from E. coli.

In one embodiment, the O-antigen comprises the structure DGIc-(a1-4)-DGal-(a1-3)-DGIcNAc.

In one embodiment, the O-antigen is an O-antigen from E. coli O18 serotype or O111 serotype.

In one embodiment, the E. coli O18 serotype is E. coli UTI89 strain.

In one embodiment, the O-antigen is used as an adjunct treatment.

In one aspect, there is provided a method for treating a bacterial infection comprising administering to a subject a therapeutically effective amount of a composition comprising an O-antigen, wherein the administration of the O-antigen mobilises transitional premonocytes (TpMos) from a bone marrow into a peripheral blood of the subject.

In one embodiment, the bacterial infection is bacterial sepsis.

In one embodiment, the O-antigen is an antigen from E. coli.

In one embodiment, the O-antigen comprises the structure DGIc-(a1-4)-DGal-(a1-3)-DGIcNAc.

In One embodiment, the O-antigen is an O-antigen from E. coli O18 Serotype or O111 serotype.

In one embodiment, the E. coli O18 serotype is E. coli UTI89 strain.

In one embodiment, the amount of O-antigen present in the composition is between 40 ng and 400 ng.

In one embodiment, the O-antigen is used as an adjunct treatment.

In one aspect, there is provided a method of mobilizing transitional premonocytes (TpMos) from a bone marrow into a peripheral blood of a subject, the method comprising administering to the subject a therapeutically effective amount of an O-antigen.

In one embodiment, the therapeutically effective amount of the O-antigen is between 40 ng and 400 ng of the O-antigen.

In one embodiment, the O-antigen is an O-antigen from E. coli.

In one embodiment, the O-antigen comprises the structure DGIc-(a1-4)-DGal-(a1-3)-DGIcNAc.

In one embodiment, the O-antigen is an O-antigen from E. coli O18 serotype or O111 serotype.

In one embodiment, the E. coli O18 serotype is E. coli UT189 strain.

In another aspect, there is provided a method of determining a prognosis of a subject having sepsis induced by a Gram-negative bacterial infection, the method comprising detecting a presence of transitional premonocytes (TpMos) in a blood sample obtained from the subject, wherein the presence of TpMos in the blood sample is indicative of a favourable clinical outcome. In the context of therapy, a “favourable clinical outcome” may mean susceptible to treatment.

The term “prognosis” is meant to include a prediction of the probable course and outcome of a clinical condition or disease. The term “prognosis” does not refer to the ability to predict the course or outcome of a condition with 100% accuracy. Instead, the term “prognosis” refers to the probability that a certain course or outcome will occur in a subject exhibiting a given condition, when compared to those individuals not exhibiting the condition. A “prognosis” can be made regarding one or more clinical outcomes, for example, the rate of progression of the disease in the subject, severity of the disease, survival rate, survival time, or response to a therapeutic intervention. Examples of a “favourable clinical outcome” include a recovery from the disease, a recovery from the disease resulting in disease free survival, a lower chance of relapse of the disease, a slower progression of the disease, a lower severity of the disease, a higher survival rate, a longer survival time, and a positive response to a therapeutic intervention in the subject.

In one embodiment of the present disclosure, administration of the specific O-antigen glycan structure mobilises TpMos into the circulation to perform the TpMo effector functions such as replenishing the loss of macrophages. The glycan structure is present on an intact O-antigen structure of LPS. The presence of TpMos is indicative of bacterial infection caused by bacterial strains with the specific O-antigen glycan structure. Nevertheless, due to the protective functions of TpMos such as by serving as a source for macrophage replenishment and by balancing proinflammatory cytokine response, the presence of TpMos helps to ameliorate bacterial infection caused by both Gram-positive and Gram-negative bacteria.

In one embodiment, TpMo mobilisation helps in modulating the immune response towards bacterial infection and sepsis by rapidly replacing the resident macrophage pool that has been depleted by bacteria-induced cell death and dampening the cytokine storm induced by inflammatory monocytes.

In one embodiment, the O-antigen may alter the immune response associated with sepsis by supporting the viability of macrophages at the site (through mobilisation of TpMos into the circulation). This results in increased control of the bacterial infection and reduction in the inflammatory response.

The O-antigen can be used as an adjunct treatment. Preferably, administration of O-antigens may be carried out alongside other main treatments during early sepsis or as soon as sepsis is diagnosed. Examples of main/primary treatments include antibiotics treatment, administration of intravenous fluid or vasopressor, and corticosteroid treatment.

In one embodiment, mobilised TpMos and TpMo-derived macrophages may increase IL-10 production in a subject having sepsis. IL-10 production may be increased during early sepsis as one of the protective mechanisms.

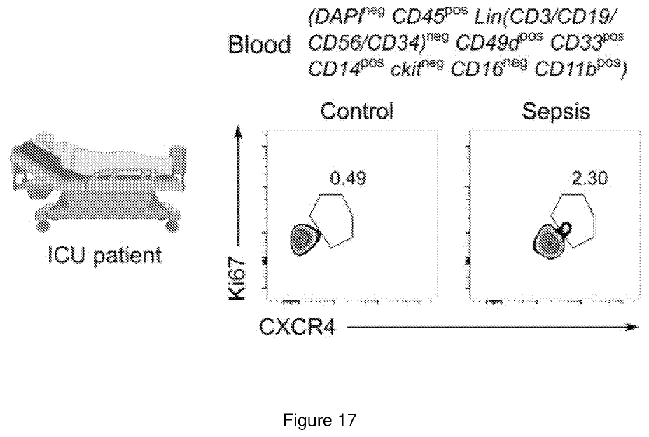

TpMos can be used as a biomarker for sepsis in that TpMos can be used to determine the prognosis of a subject with sepsis. The presence of TpMos improves sepsis survival and is thus indicative of a favourable clinical outcome. This is evident by data obtained from the setup shown in FIG. 17. Some septic patients from ICU have TpMos in the circulation. Patients without TpMos in the circulation compromised sometime later, while patients who managed to be discharged (and hence, recover or achieved favourable clinical outcomes) retained TpMos in the circulation.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### MATERIALS AND METHODS

### Tissue Preparation for Flow Cytometry and Sorting

A total of 200 μl of blood was obtained via an incision in the submandibular region using a 5-mm lancet and treated with commercial-grade red blood cell lysis buffer (eBioscience). Mice were sacrificed by CO₂ inhalation. To harvest peritoneal cavity cells, 3 ml of PBS with 2 mM EDTA was administered into the peritoneum using a 26 1/2-gauge needle. The PL was collected, and the cell suspension was passed through a 70-μm nylon mesh. To collect BM cells, mouse femurs were flushed using a 23-gauge needle in PBS with 2 mM EDTA and 3% fetal bovine serum, and the effluent was passed through a 70-μm nylon mesh. Antibodies were purchased from eBioscience, Biolegend, BD Biosciences, or R&D Systems. Mouse cells were stained with the following antibodies: CCR2 (475301), CD3e (145-2C11), CD11b (M1-70), CD11c (N418), CD16/32 (2.4G2), CD31(390), CD43 (S7), CD45 (30-F11), CD45.1 (A20), CD45.2 (104), CD45R (B220) (RA3-6B2), CD62L (MEL-14), CD90.2 (53-2.1), CD106 (429/MVCAM. A), CD115 (AFS98), c-kit (2B8), CXCR4 (2B11), CX3CR1 (SA011F11), F4/80 (BM8), I-A/I-E (M5/114.15.2), Flt-3 (A2F10), Ly6C (HK1.4), Ly6G (1A8), NK1.1 (PK136), PD-L1 (10F.9G2), Sca-1 (D7), Siglec-F (E50-2440), Tim-4 (54). Dead cells were identified and excluded using 4′,6-diaminido-2-phenylindole (DAPI) staining. Blood and BM monocytes were identified as Lin (B220/CD90.2/NK1.1)^{neg} Ly6G^{neg} CD115^{pos}. Monocytes were further divided into cMoPs (CD11b^{neg} Ly6C^{hi} ckit^{pos}), TpMos (CD11b^{lo} Ly6C^{hi} ckit^{neg} CXCR4^{hi}), and MatMos (CD11b^{pos} Ly6C^{hi} ckit^{neg} CXCR4^{lo}). Total PL macrophages were identified as CD45^{pos} Lin (B220/CD90.2/NK1.1)^{neg} CD11^{bpos} F4/80^{pos}. To check for cytokine expression, BM or peritoneal cells were stimulated with LPS for 3 hours (or 24 hours for iNOS expression) at 37° C., 5% CO₂ in the presence of GolgiStop and GolgiPlug (BD Biosciences) in the medium at a dilution recommended by the manufacturer's instructions; stained with relevant surface markers; fixed with fixation/permeabilization buffer (BD Biosciences) before staining with IL-1β (NJTEN3), IL-6 (MQ213A5), IL-10 (JES5-16E3), TNF-α (MP6-XT22), and iNOS/NOS2 (CXNFT); washed; and acquired via flow cytometry. Cells were acquired on BD LSR II flow cytometer using FACSDiva software, and data were subsequently analyzed with FlowJo software (Tree Star). The total number of cells collected was quantified using count beads (CountBright; Life Technologies) according to the manufacturer's protocol. BM TpMos and MatMos were sorted using a BD FACSAria II sorter to achieve >98% purity.

### Adoptive Transfer of TpMos and MatMos

BM TpMos and MatMos sorted from WT or Fucci-474 mice were suspended in 200 μl of PBS and adoptively transferred through intravenous or intraperitoneal routes (as indicated) into CD45.1 recipient mice in steady state or after induction of sepsis. Recipient mice were euthanized at the time points indicated, and blood, BM, and PL were harvested, surface stained, and analyzed by flow cytometry. For intra-BM (IBM) transfer of BM TpMos and MatMos, sorted 5×10⁵ GFP⁺ TpMos and tdTomato⁺ MatMos were resuspended in a 1:1 ratio and transferred as a single injection into CD45.2⁺mice. Briefly, recipient mice were anesthetized using ketamine/xylazine with legs shaven before transfer. Recipient tibia and blood were collected, surface-stained, and analyzed by flow cytometry 9 hours after bacterial infection.

### Bacterial Infection Model

Escherichia coli (E. coli) strain UTI89, DFB1655 L5 (L5), DFB1655 L9(L9 ) were provided by Swaine Chen, A*STAR Singapore. E. coli MG1655 strain, Acinetobacter baumannii, Pseudomonas aeruginosa, Staphylococcus aureus, Enterococcus faecalis, and Enterococcus faecium were provided by Ghee Chuan Lai, A*STAR Singapore. To prepare for infection, bacteria were cultured in lysogeny broth (LB) and incubated in a shaker incubator at 37° C. Bacteria were grown to mid-log phase [an optical density at 600 nm (OD₆₀₀) of 0.4 to 0.6], and then washed twice with phosphate-buffered saline (PBS). Mice were infected intraperitoneally with 1×10⁶ colony-forming units (CFU) E. coli in 200 μL of PBS. Control group was injected intraperitoneally with 200 μL of PBS. Mice were harvested 9 or 18 hours after infection. E. coli burden was measured by collecting the peritoneal fluid 18 hours after infection and diluted accordingly before culturing overnight on LB plates. Colonies were counted, and CFU calculated the next day. Bar graphs were plotted as CFU per microliter of peritoneal fluid.

### CLP-Induced Sepsis

Experimental procedures were performed as previously described (Rittirsch et al., 2009, Nature Protocols). The peritoneal cavity was exposed under ketamine/xylazine anesthesia, and the cecum was exteriorized. Mid-and high-grade sepsis were performed through 50 and 75% cecum ligation, respectively, using a nonabsorbable 7-0 suture. A 26 ½ -gauge needle was used to perforate the distal end of the cecum, and a small drop of feces was extruded through the puncture before being relocated back into the peritoneal cavity. The peritoneum was closed and subsequently treated with saline and buprenorphine via subcutaneous injection. Age-and gender-matched sham-operated controls were included for all procedures.

### BrdU Pulsing and Proliferation Assays

Mice were administered 1.5 mg BrdU (BD Biosciences) via intraperitoneal injection for 30 min to assess their proliferative capacity. To detect BrdU incorporation, cells were surface-stained, fixed, permeabilized, and subjected to intracellular staining with fluorescein isothiocyanate-or anaphase-promoting complex-conjugated anti-BrdU antibody according to the manufacturer's protocol (BrdU Flow kit; BD Biosciences) before analysis by flow cytometry. The Fucci-transgenic mouse model, which labels cells in the S-G₂-M phase of the cell cycle with a green-emitting fluorescent protein, was also used to identify proliferating cells through flow cytometry.

### EXAMPLES

Non-limiting examples of the invention and comparative examples will be further described in greater detail by reference to specific Examples, which should not be construed as in any way limiting the scope of the invention.

Teh, Ye Chean, et al. “Transitional Premonocytes Emerge in the Periphery for Host Defense Against Bacterial Infections.” Science Advances, vol. 8, no. 9, American Association for the Advancement of Science (AAAS), Mar. 2022. is hereby incorporated by reference in its entirety.

### Example 1

Tissue resident macrophages (TRMs) act as early immune sentinels for host defense. However, they are highly susceptible towards cellular death during inflammation and infections. While a temporary loss of TRMs does not impose immediate danger to the host in sterile conditions, the niche comprised of dying macrophages in the presence of pathogenic bacteria predisposes the host to increased morbidity if these cells are not effectively replaced. Hence, monocytes must be recruited rapidly into the empty niche to defend against these fast-replicating bacteria. Other than fulfilling their role as an emergency reservoir of macrophages, monocytes are also able to execute effector functions such as phagocytosis and cytokine production to reduce the bacterial load. However, execution of effector functions of monocytes often leads to their exhaustion followed by cell death, which limits their capacity for subsequent differentiation into macrophages in these scenarios.

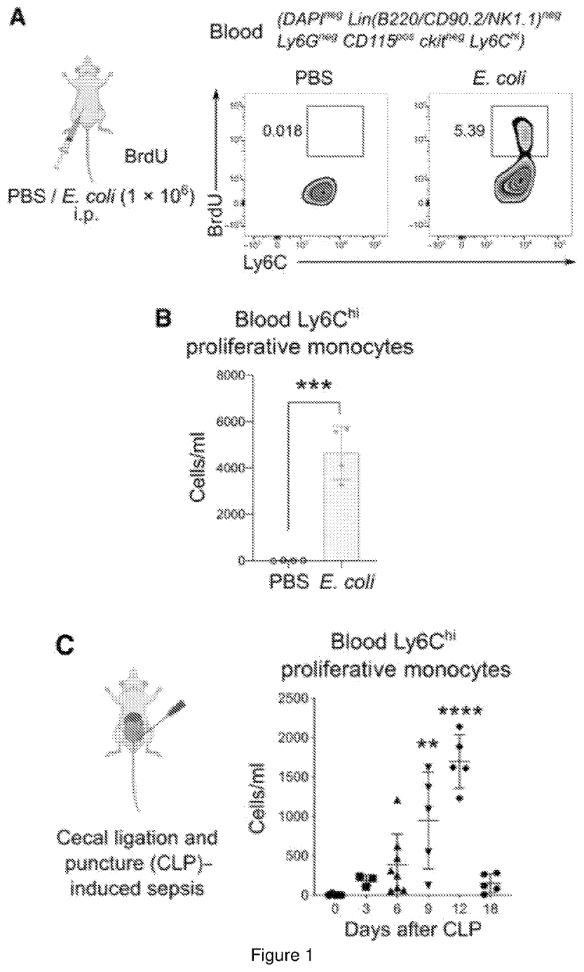

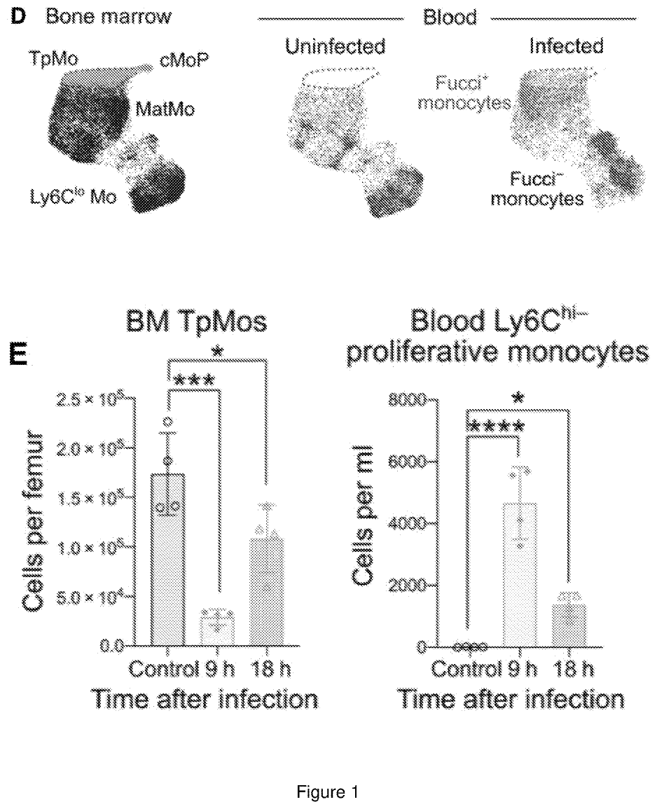

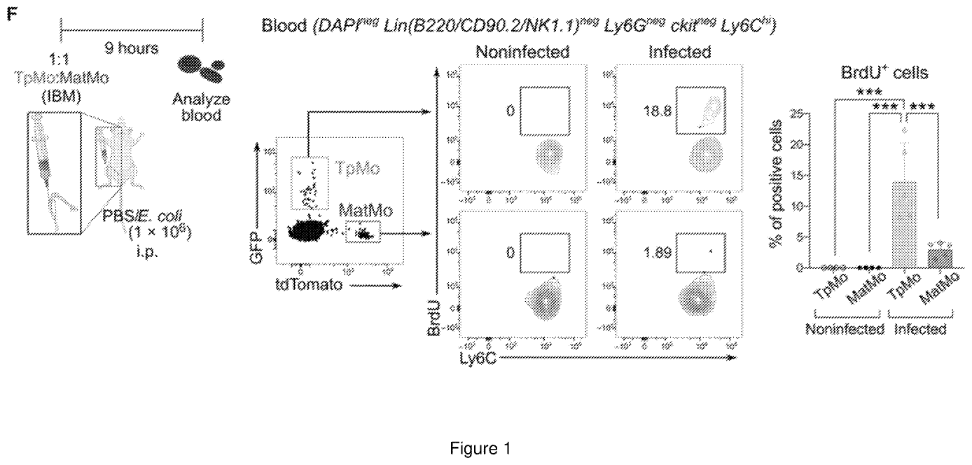

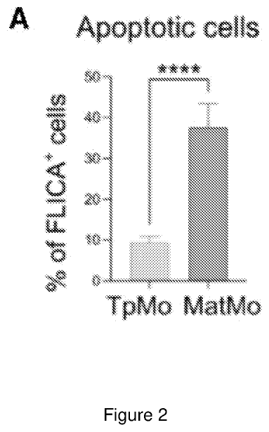

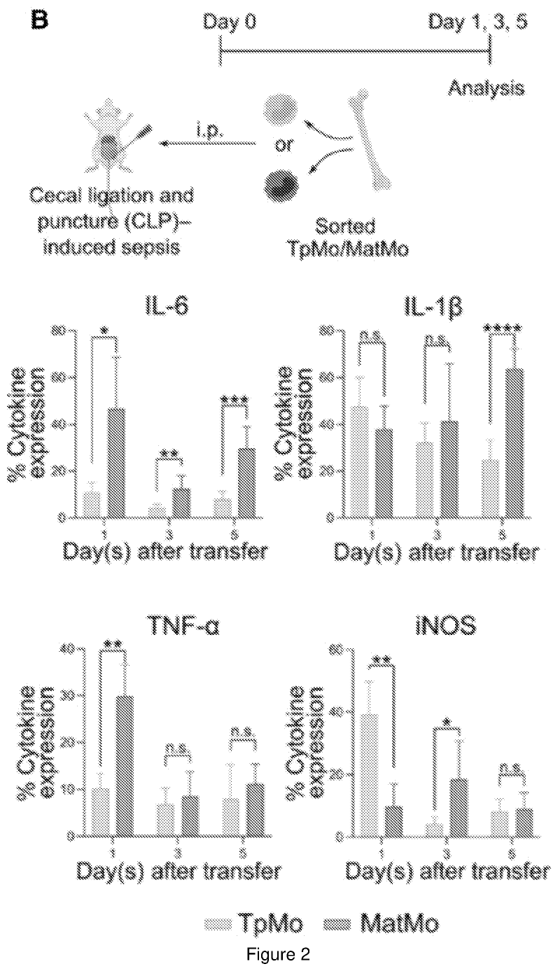

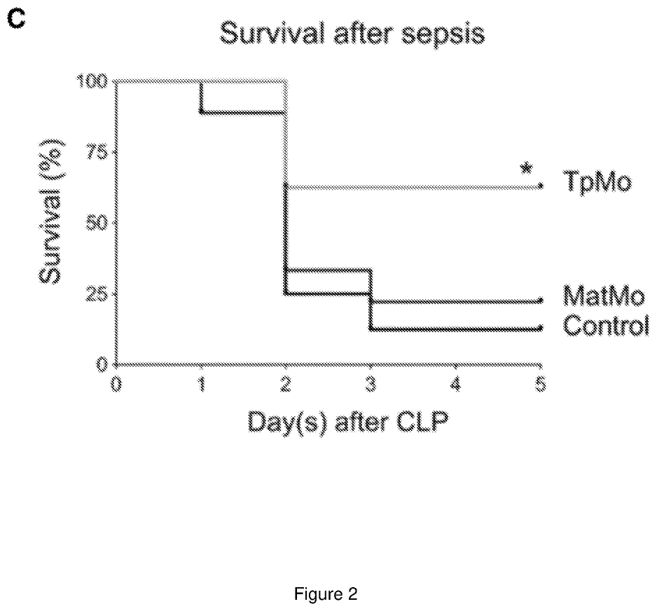

The inventors elucidated the differential roles of monocyte subsets during bacterial infection and sepsis, highlighting a novel protective response of TpMos towards bacterial infection in the periphery. Specifically, a population of Ly6C^{hi} proliferative monocytes was detected in the circulation of E. coli-infected and CLP-induced septic mice (FIG. 1A-C). Through UMAP analysis and adoptive transfer experiments, these proliferative monocytes were identified as TpMos from the bone marrow (BM) (FIG. 1D-F). TpMos would then infiltrate inflamed tissues to repopulate the empty niche left behind by dying TRMs during bacterial infection and sepsis. Other than fulfilling their role as precursors to replenish the macrophage pool during severe inflammation induced by bacterial infection, TpMos could also execute effector functions. They were found to be less inflammatory and less susceptible to apoptosis in response to CSF-1 signalling (FIG. 2A, B). Importantly, TpMos could confer protection to septic mice by balancing the pro-inflammatory response associated with the cytokine storm (FIG. 2C).

### Example 2

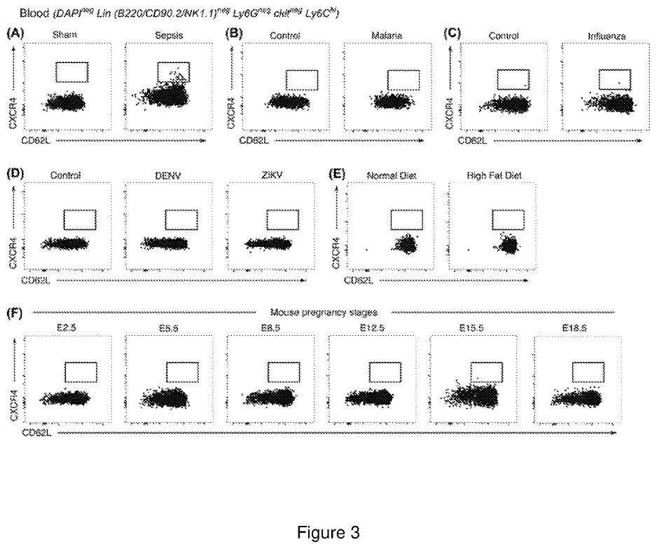

It was investigated whether TpMos could also be mobilised under other inflammatory conditions. Interestingly, TpMos were only detected under bacteria exposure (FIG. 3A) and not in tested parasitic or viral infection models such as malaria (FIG. 3B), influenza (FIG. 3C), dengue, and zika (FIG. 3D). TpMo mobilization was not detected in non-infectious disease settings such as the high fat diet model (FIG. 3E) and pregnancy model (FIG. 3F).

### Example 3

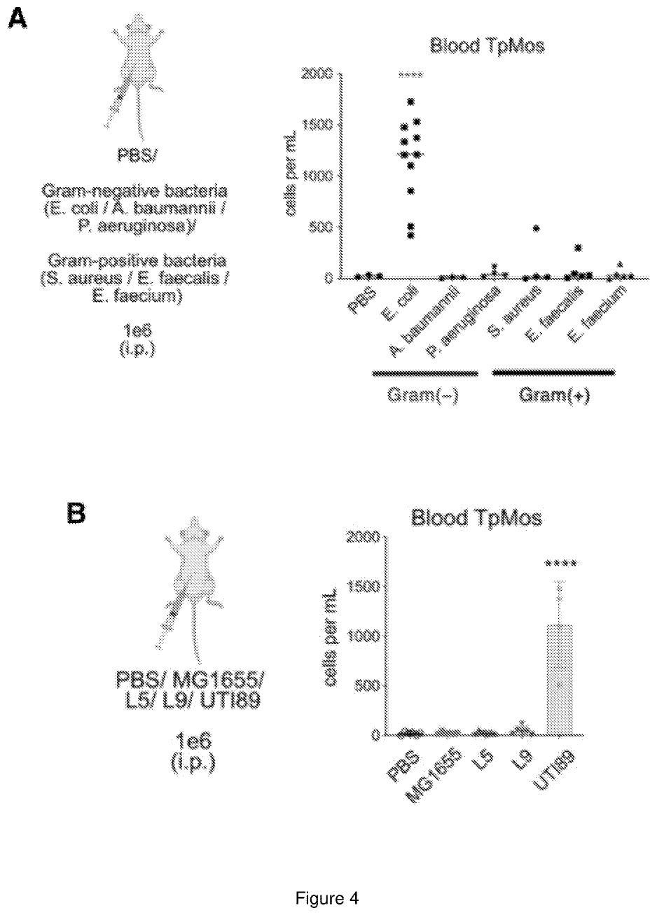

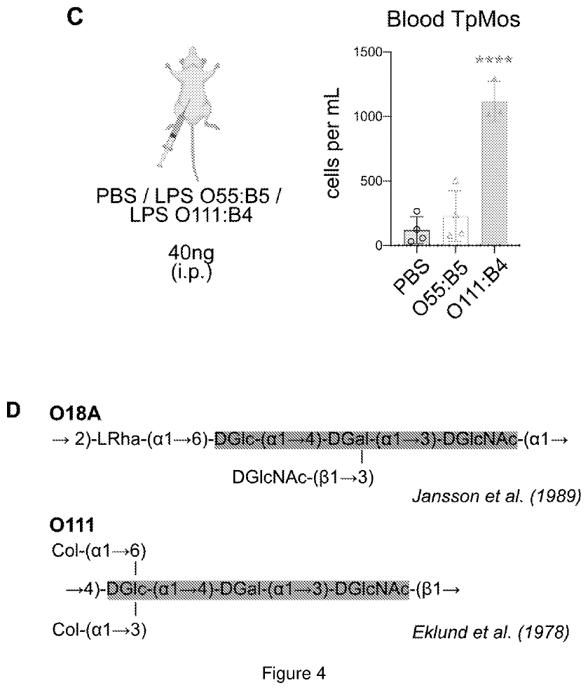

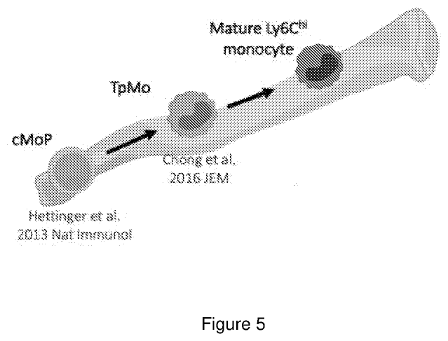

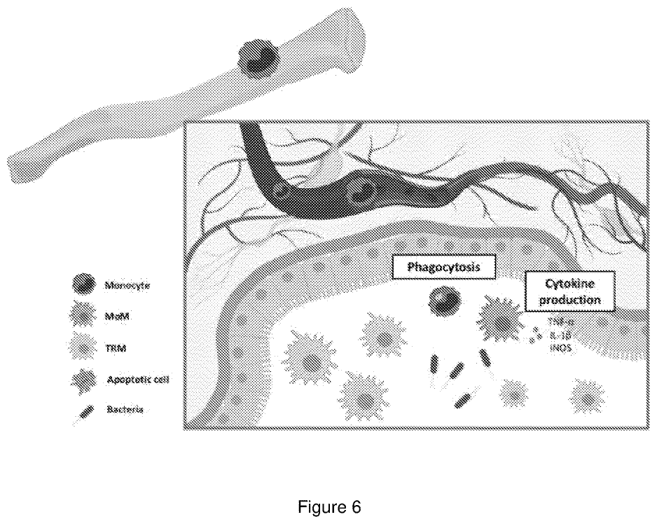

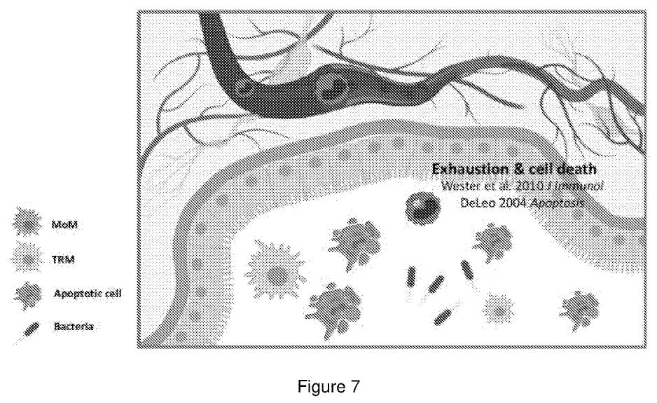

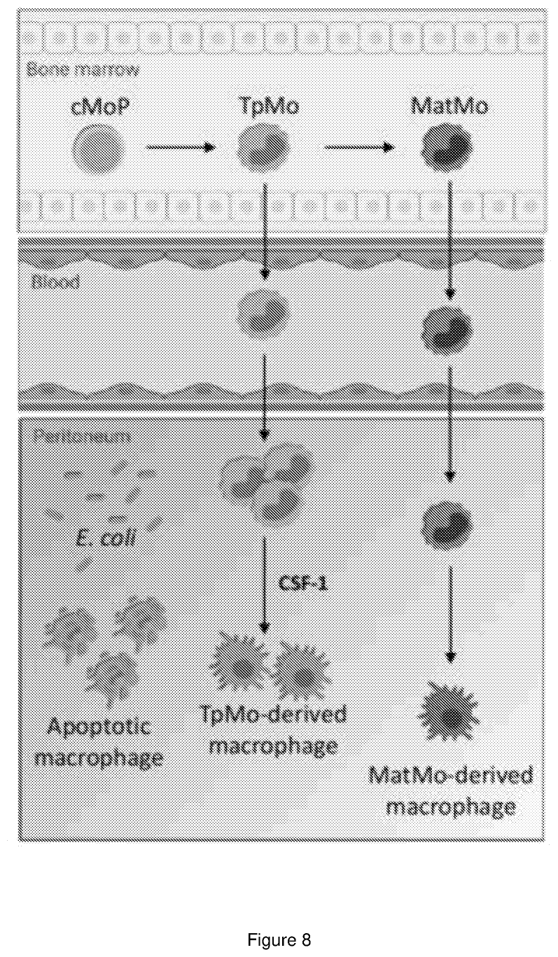

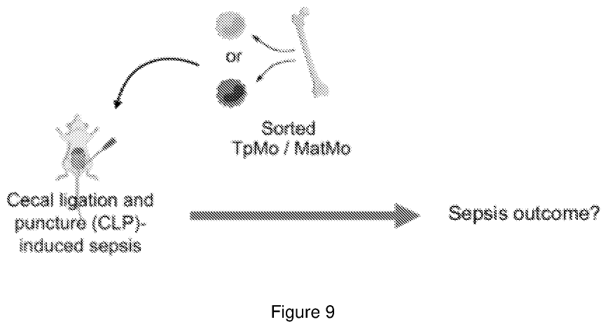

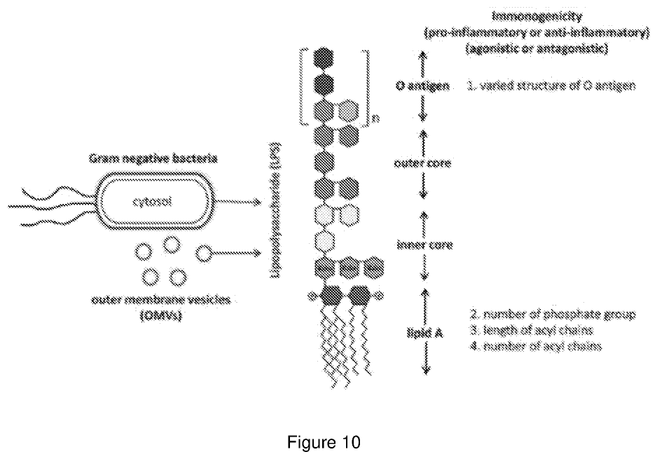

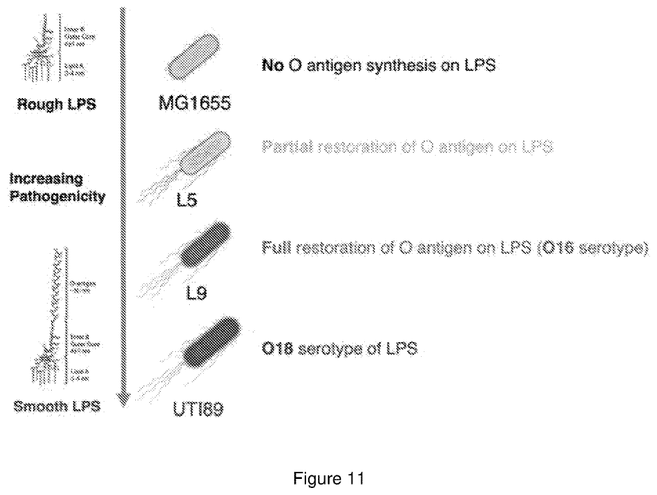

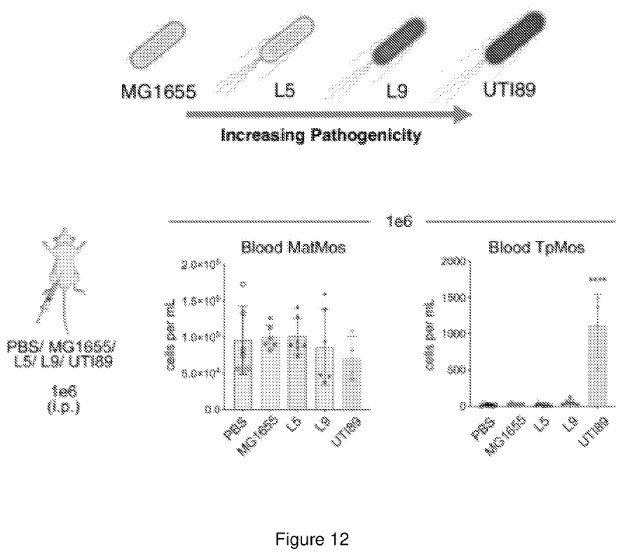

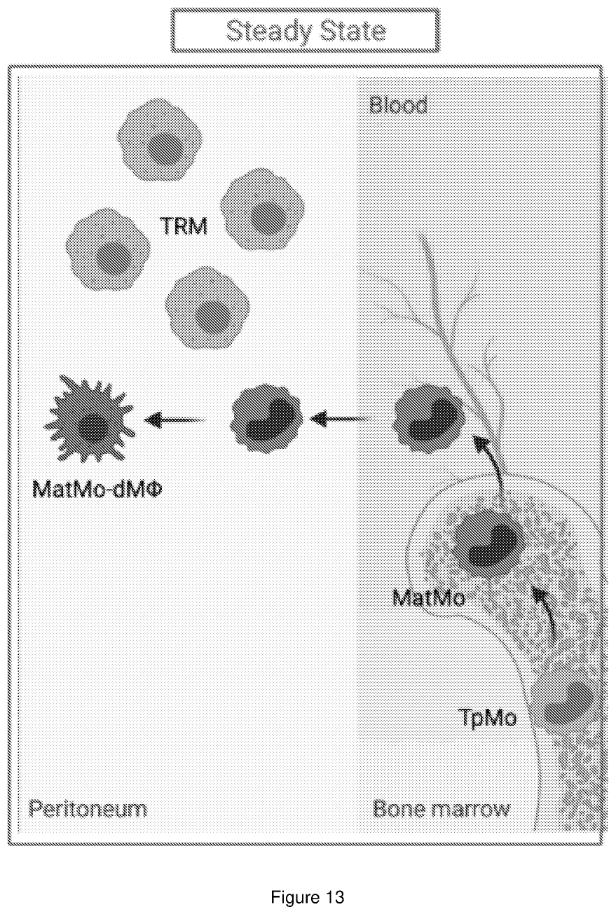

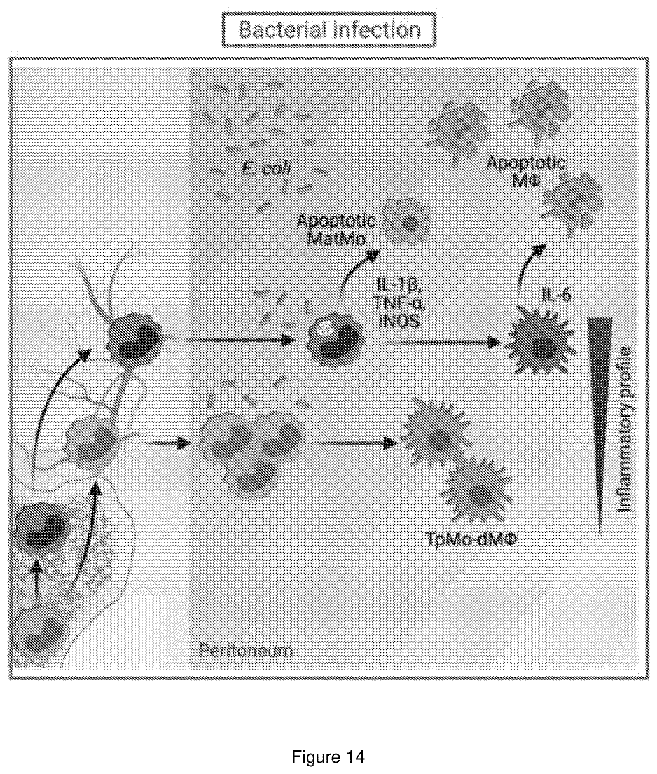

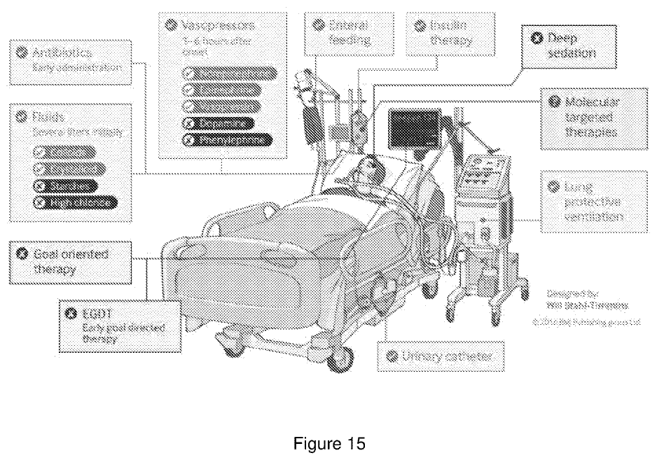

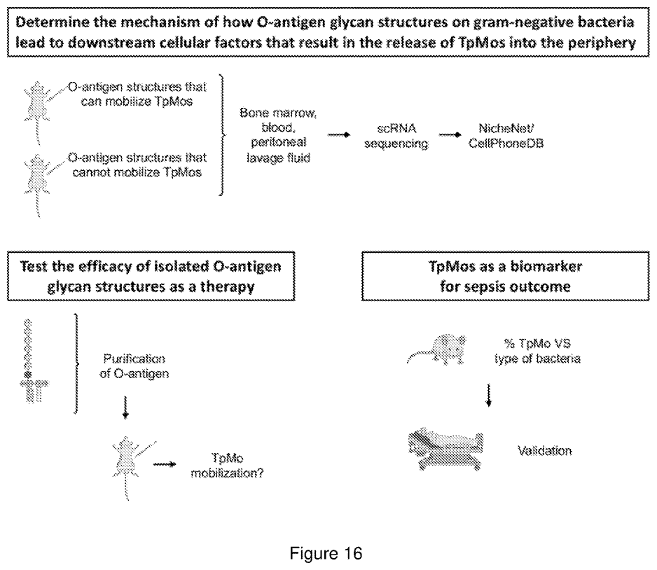

Since TpMos were only detected in the circulation of mice exposed to bacteria, it was next determined whether certain bacteria properties trigger the release of TpMos. It has been reported that a mixture of bacteria was commonly found in the blood cultures of septic patients. The CLP-induced sepsis model in the present study mimics human polymicrobial sepsis as it relies on the bacteria in the mouse cecum to exert inflammatory conditions. However, there is a diversity of bacteria in the mouse microbiota and different bacterial species and components are recognized by the host distinctively. Since there is a mixture of gram-positive and gram-negative bacteria in the mouse microbiota, the inventors wanted to determine if all bacteria are involved in triggering TpMo egress. Interestingly, among the bacteria tested, only the gram-negative E. coli bacteria triggered a substantial number of TpMos into the circulation (FIG. 4A), suggesting that their mobilization was triggered by signals likely restricted to gram-negative bacteria.

To determine the specific factors resulting in this phenomenon, the inventors looked into lipopolysaccharide (LPS) which represents a major part of the cell wall component of gram-negative bacteria. LPS comprises of Lipid A, core oligosaccharide, and O-polysaccharide (O-antigen). Interestingly, TpMos were only mobilized upon E. coli infection with an O18 serotype (UTI89). MG1655, a laboratory strain of E. coli without the ability to synthesize O-antigen, was unable to trigger the mobilization of TpMos even after restoration of its wildtype O16 structure in different doses (L5: partial restoration, L9: full restoration) (FIG. 4B). also It was also confirmed that TpMo mobilization was restricted to the O-antigen serotype as the presence of these cells could also be detected with administration of LPS alone. However, while LPS of the O111 serotype was able to mobilize TpMos, LPS of the O55 serotype was unable to (FIG. 4C), even though both serotypes have been documented to be enteropathogenic strains. Preliminary analysis of the O-antigen structures of O18 and O111, which induced the greatest number of TpMos into the circulation, revealed a common glycan structure as indicated in FIG. 4D, suggesting that certain O-antigen glycan structures were able to induce the release of TpMos into the circulation.

Taken together, data from the present study shows that TpMos have protective functions during bacterial infection and sepsis, and their mobilization is specifically induced by gram-negative bacteria with certain O-antigen glycan structures, suggesting the potential of utilizing O-antigens as adjuvants or intervention strategy to mobilize TpMos during sepsis to improve patient outcomes.

### Example 4

To determine if TpMos can be potentially used as a biomarker for sepsis outcome, blood samples were collected from septic patients in ICU to check for the presence of TpMos. The preliminary data shows that patients without TpMos in their circulation compromised some time later, while some patients who managed to be discharged retained TpMos in their circulation as shown in FIG. 17, suggesting that the presence of TpMos indicates a favourable clinical outcome, demonstrating the potential of using TpMos a biomarker for sepsis outcome. TpMos are absent in the circulation of healthy patients, only present in the bone marrow during steady state. Thus, any amount of TpMos above zero in the circulation during bacterial infection/sepsis would be indicative of a favourable clinical outcome for septic patients.

While embodiments of the invention have been particularly shown and described with reference to specific embodiments, it should be understood by those skilled in the art that various changes in form and detail may be made therein without departing from the scope of the invention as defined by the appended claims. The scope of the invention is thus indicated by the appended claims and all changes which come within the meaning and range of equivalency of the claims are therefore intended to be embraced.

1. Use of an O-antigen in the manufacture of a medicament for treating a bacterial infection, wherein the medicament to be administered mobilises transitional premonocytes (TpMos) from a bone marrow into a peripheral blood of a subject.

2. The use of claim 1, wherein the bacterial infection is bacterial sepsis.

3. The use of claim 2, wherein the O-antigen is an O-antigen from E. coli.

4. The use of any one of claims 1 to 3, wherein the O-antigen comprises the structure DGIc-(a1-4)-DGal-(a1-3)-DGIcNAc.

5. The use of any one of claims 1 to 4, wherein the O-antigen is an O-antigen from E. coli O18 serotype or O111 serotype.

6. The use of claim 5, wherein the E. coli O18 serotype is E. coli UTI89 strain.

7. The use of any one of claims 1 to 6, wherein the medicament is used as an adjunct treatment.

8. A pharmaceutical composition comprising (a) a therapeutically effective amount of an O-antigen comprising the structure DGIc-(a1-4)-DGal(a1-3)-DGIcNAc; and (b) one or more pharmaceutically acceptable carriers and/or diluents.

9. The pharmaceutical composition of claim 8, wherein the O-antigen is an O-antigen from E. coli.

10. The pharmaceutical composition of claims 8 or 9, wherein the O-antigen is an O-antigen from E. coli O18 serotype or O111 serotype.

11. The pharmaceutical composition of claim 10, wherein the E. coli O18 serotype is E. coli UT189 strain.

12. An O-antigen for use in treating a bacterial infection, wherein the O-antigen mobilises TpMos from a bone marrow into a peripheral blood of a subject.

13. The O-antigen of claim 12, wherein the bacterial infection is bacterial sepsis.

14. The O-antigen of claims 12 or 13, wherein the O-antigen is an O-antigen from E. coli.

15. The O-antigen of any one of claims 12 to 14, wherein the O-antigen comprises the structure DGIc-(a1-4)-DGal(a1-3)-DGIcNAc.

16. The O-antigen of any one of claims 12 to 15, wherein the O-antigen is an O-antigen from E. coli O18 serotype or O111 serotype.

17. The O-antigen of claim 16, wherein the E. coli O18 serotype is E. coli UTI89 strain.

18. The O-antigen of any one of claims 12 to 17, wherein the O-antigen is used as an adjunct treatment.

19. A method for treating a bacterial infection comprising administering to a subject a therapeutically effective amount of a composition comprising an O-antigen, wherein the administration of the O-antigen mobilises transitional premonocytes (TpMos) from a bone marrow into a peripheral blood of the subject.

20. The method of claim 19, wherein the bacterial infection is bacterial sepsis.

21. The method of claims 19 or 20, wherein the O-antigen is an O-antigen from E. coli.

22. The method of any one of claims 19 to 21, wherein the O-antigen comprises the structure DGIc-(a1-4)-DGal(a1-3)-DGIcNAc.

23. The method of any one of claims 19 to 22, wherein the O-antigen is an O-antigen from E. coli O18 serotype or O111 serotype.

24. The method of claim 23, wherein the E. coli O18 serotype is E. coli UTI89 strain.

25. The method of any one of claims 19 to 24, wherein the amount of O-antigen present in the composition is between 40 ng and 400 ng.

26. The method of any one of claims 19 to 25, wherein the O-antigen is used as an adjunct treatment.

27. A method of mobilizing transitional premonocytes (TpMos) from a bone marrow into a peripheral blood of a subject, the method comprising administering to the subject a therapeutically effective amount of an O-antigen.

28. The method of claim 27, wherein the therapeutically effective amount of the O-antigen is between 40 ng and 400 ng of the O-antigen.

29. The method of claims 27 or 28, wherein the O-antigen is an O-antigen from E. coli.

30. The method of any one of claims 27 to 29, wherein the O-antigen comprises the structure DGIc-(a1-4)-DGal(a1-3)-DGIcNAc.

31. The method of any one of claims 27 to 30, wherein the O-antigen is an O-antigen from E. coli O18 serotype or O111 serotype.

32. The method of claim 31, wherein the E. coli O18 serotype is E. coli UTI89 strain.

33. A method of determining a prognosis of a subject having sepsis induced by a Gram-negative bacterial infection, the method comprising detecting a presence of transitional premonocytes (TpMos) in a blood sample obtained from the subject, wherein the presence of TpMos in the blood sample is indicative of a favourable clinical outcome.
